(19) United States
(12) Patent Application Publication
(10) Pub. No.: US 2025/0391551 A1
(43) Pub. Date: Dec. 25, 2025

(54) SYSTEM AND METHOD FOR MANAGING SURGICAL HARDWARE

(71) Applicant: ORTHOSOFT ULC, Montreal (CA)

(72) Inventors: Pierre COUTURE, Montreal (CA); Louis-Philippe AMIOT, Montreal (CA)

(21) Appl. No.: 19/243,361

(22) Filed: Jun. 19, 2025

Related U.S. Application Data
(60) Provisional application No. 63/664,059, filed on Jun. 25, 2024.

(51) Int. Cl. G16H 40/40; A61B 34/20; A61B 34/32; A61B 90/00; B25J 9/16; G06T 7/73; G06V 20/50; G16H 40/20

(52) U.S. Cl. CPC G16H 40/40; A61B 34/32; B25J 9/161; G06T 7/74; G06V 20/50; G16H 40/20; A61B 2034/2055; A61B 2034/2065; A61B 2090/0805; G06T 2207/10016; G06T 2207/30004; G06T 2207/30204; G06V 2201/034

(57) ABSTRACT

A system for managing surgical hardware in computer-assisted surgery may have a processing unit; and a non-transitory computer-readable memory communicatively coupled to the processing unit and comprising computer-readable program instructions executable by the processing unit for: obtaining at least one post-surgery image of a plurality of instruments used in a surgical procedure, after the surgical procedure is completed and before the plurality of instruments are stored in a storage device; processing the at least one post-surgery image to identify the instruments of the plurality; and outputting data relating to a position of at least one of the instruments in the storage device to assist a user in storing the at least one instrument.

### CROSS-REFERENCE TO RELATED APPLICATION

The present application claims the priority of U.S. Patent Application No. 63/664,059, filed on Jun. 25, 2024, the content of which is incorporated hereby reference.

### TECHNICAL FIELD

The present application relates to computer-assisted surgery and to management of surgical hardware.

### BACKGROUND OF THE ART

The navigation of surgical instruments or tools is an integral part of computer-assisted surgery (hereinafter “CAS”). The tools are navigated, i.e., tracked for position and/or orientation, in such a way that relative information pertaining to bodily parts is obtained. The information may be used in various interventions (e.g., orthopedic surgery, trauma surgery, neurological surgery) with respect to the body, such as bone alterations, implant positioning, incisions and the like during surgery.

In a single surgical procedure, numerous tools may be used depending on the type of intervention. For example, in the context of orthopedic surgery, drills, awls, reamers, pointers, etc, are examples of the hundreds of available tools, that may come in different sizes and/or configuration. Typically, such tools are metallic tools, that are then recuperated, to be sterilized, for subsequent use for another patient. Accordingly, at the outset of a surgical procedure, the operating team may have access to a large number of tools, typically arranged in trays or like supports. For example, in some types of orthopedic surgeries, multiple trays may be simultaneously in the operating room, available to the operating team, At the conclusion of a surgical procedure, the used tools are typically gathered randomly in a bucket or placed in sterilization trays, for sterilization.

As a result, hospitals, clinics and like medical facilities may find themselves with disorganized tool sets, with challenges in maintaining inventory, in properly organizing tool storage, among other potential issues. An important challenge is the cost, time and risk of error related to identifying and sorting instruments in cleaning/sterilization trays.

### SUMMARY

In accordance with a first aspect of the present disclosure, there is provided a system for managing surgical hardware in computer-assisted surgery, comprising: a processing unit; and a non-transitory computer-readable memory communicatively coupled to the processing unit and comprising computer-readable program instructions executable by the processing unit for: obtaining at least one post-surgery image of a plurality of instruments used in a surgical procedure; processing the at least one post-surgery image to identify the instruments of the plurality; and outputting data relating to a position of at least one of the instruments in a storage device to assist a user in storing the at least one instrument.

Further in accordance with the first aspect, for instance, the computer-readable program instructions are executable by the processing unit for obtaining a list of the plurality of instruments from a computer-assisted surgery system operating a surgical workflow during the surgical procedure.

Still further in accordance with the first aspect, for instance, the computer-readable program instructions are executable by the processing unit for comparing the identified instruments from the plurality to the list of instruments used during the surgical procedure.

Still further in accordance with the first aspect, for instance, the computer-readable program instructions are executable by the processing unit for outputting data relating to any discrepancy between the list and the plurality of instruments.

Still further in accordance with the first aspect, for instance, the computer-readable program instructions are executable by the processing unit for obtaining the at least one post-surgery image of the plurality of instruments used in the surgical procedure in the storage tray, and for outputting data relating to any discrepancy between a current position and a set position of at least one of the plurality of instruments.

Still further in accordance with the first aspect, for instance, the computer-readable program instructions are executable by the processing unit for outputting said data relating to the position of at least one of the instruments in the storage device as instructions for driving a robot configured to store the at least one instrument in the storage device.

Still further in accordance with the first aspect, for instance, the computer-readable program instructions are executable by the processing unit for driving the robot to store the at least one instrument in the storage device.

Still further in accordance with the first aspect, for instance, the computer-readable program instructions are executable by the processing unit for obtaining a video feed when obtaining the at least one post-surgery image of the plurality of instruments.

Still further in accordance with the first aspect, for instance, the computer-readable program instructions are executable by the processing unit for processing the video feed when processing the at least one post-surgery image.

Still further in accordance with the first aspect, for instance, the computer-readable program instructions are executable by the processing unit for obtaining the at least one post-surgery image of the plurality of instruments in a sterilization tray.

Still further in accordance with the first aspect, for instance, the computer-readable program instructions are executable by the processing unit for obtaining the at least one post-surgery image of the plurality of instruments in the sterilization tray prior to sterilization.

Still further in accordance with the first aspect, for instance, the computer-readable program instructions are executable by the processing unit for obtaining the at least one post-surgery image of the plurality of instruments in the sterilization tray after sterilization.

Still further in accordance with the first aspect, for instance, the computer-readable program instructions are executable by the processing unit for obtaining the at least one post-surgery image of the plurality of instruments in the sterilization tray, the sterilization tray being the storage device.

Still further in accordance with the first aspect, for instance, the computer-readable program instructions are executable by the processing unit for imaging the plurality of instruments to obtain the at least one post-surgery image.

Still further in accordance with the first aspect, for instance, the computer-readable program instructions are executable by the processing unit for imaging the plurality of instruments with a non-radiographic digital camera.

Still further in accordance with the first aspect, for instance, the computer-readable program instructions are executable by the processing unit for imaging the plurality of instruments with the non-radiographic digital camera being head-mounted to a head of a user.

Still further in accordance with the first aspect, for instance, the computer-readable program instructions are executable by the processing unit for recognizing an optical code on at least one of the instruments when processing the at least one post-surgery image to identify the instruments of the plurality, and for retrieving the identity of the instrument from the optical code.

Still further in accordance with the first aspect, for instance, the computer-readable program instructions are executable by the processing unit for comparing images of the instruments to images of instruments in a database of instrument images when processing the at least one post-surgery image to identify the instruments of the plurality.

Still further in accordance with the first aspect, for instance, the computer-readable program instructions are executable by the processing unit for obtaining at least one post-surgery image of a plurality of instruments used in the surgical procedure after the surgical procedure is completed and before the plurality of instruments are stored in a storage device.

Still further in accordance with the first aspect, for instance, the computer-readable program instructions are executable by the processing unit for outputting an image of the at least one instrument when outputting said data.

Still further in accordance with the first aspect, for instance, the computer-readable program instructions are executable by the processing unit for outputting an image of the at least one instrument as currently laid, with a visual marker on the image.

Still further in accordance with the first aspect, for instance, the computer-readable program instructions are executable by the processing unit for outputting a mixed reality image of the at least one instrument with the visual marker.

Still further in accordance with the first aspect, for instance, the computer-readable program instructions are executable by the processing unit for outputting an image of the storage device with a visual marker on said position when outputting said data.

Still further in accordance with the first aspect, for instance, the computer-readable program instructions are executable by the processing unit for outputting a mixed reality image of the storage device with the visual marker.

In accordance with a second aspect of the present disclosure, there is provided a system for managing surgical hardware in computer-assisted surgery, comprising: a processing unit; and a non-transitory computer-readable memory communicatively coupled to the processing unit and comprising computer-readable program instructions executable by the processing unit for: obtaining at least one post-surgery image of a plurality of instruments used in a surgical procedure, after the surgical procedure is completed; processing the at least one post-surgery image to identify the instruments of the plurality; comparing the identified instruments from the plurality to a list of instruments used during the surgical procedure; and outputting data relating to any discrepancy between the list and the plurality of instruments.

Further in accordance with the second aspect, for instance, the computer-readable program instructions are executable by the processing unit for obtaining at least one intraoperative image of a surgical tray prior to or during the surgical procedure.

Still further in accordance with the second aspect, for instance, the computer-readable program instructions are executable by the processing unit for processing the at least one intraoperative image to identify the instruments on the surgical tray.

Still further in accordance with the second aspect, for instance, the computer-readable program instructions are executable by the processing unit for generating said list of instruments from the instruments on the surgical tray.

Still further in accordance with the second aspect, for instance, the computer-readable program instructions are executable by the processing unit for obtaining said list of instruments from a computer-assisted surgery system operating a surgical workflow during the surgical procedure.

Still further in accordance with the second aspect, for instance, the computer-readable program instructions are executable by the processing unit for obtaining a video feed when obtaining the at least one post-surgery image of the plurality of instruments.

Still further in accordance with the second aspect, for instance, the computer-readable program instructions are executable by the processing unit for processing the video feed when processing the at least one post-surgery image.

Still further in accordance with the second aspect, for instance, the computer-readable program instructions are executable by the processing unit for obtaining the at least one post-surgery image of the plurality of instruments in a sterilization tray.

Still further in accordance with the second aspect, for instance, the computer-readable program instructions are executable by the processing unit for obtaining the at least one post-surgery image of the plurality of instruments in the sterilization tray prior to sterilization.

Still further in accordance with the second aspect, for instance, the computer-readable program instructions are executable by the processing unit for obtaining the at least one post-surgery image of the plurality of instruments in the sterilization tray after sterilization.

Still further in accordance with the second aspect, for instance, the computer-readable program instructions are executable by the processing unit for imaging the plurality of instruments to obtain the at least one post-surgery image.

Still further in accordance with the second aspect, for instance, the computer-readable program instructions are executable by the processing unit for imaging the plurality of instruments with a non-radiographic digital camera.

Still further in accordance with the second aspect, for instance, the computer-readable program instructions are executable by the processing unit for recognizing an optical code on at least one of the instruments when processing the at least one post-surgery image to identify the instruments of the plurality, and for retrieving the identity of the instrument from the optical code.

Still further in accordance with the second aspect, for instance, the computer-readable program instructions are executable by the processing unit for comparing images of the instruments to images of instruments in a database of instrument images when processing the at least one post-surgery image to identify the instruments of the plurality.

Still further in accordance with the second aspect, for instance, the computer-readable program instructions are executable by the processing unit for obtaining the at least one post-surgery image of the plurality of instruments used in the surgical procedure, after the surgical procedure is completed and before the plurality of instruments are stored in eth storage device.

### DESCRIPTION OF THE DRAWINGS

FIG. 1 is a block diagram of a computer-assisted surgery (CAS) system with optional head-mounted navigation in accordance a variant with the present disclosure;

FIG. 2 is a perspective view and block diagram of a head-mounted device in accordance with another variant of the present disclosure;

FIG. 3 is a flow chart of a method for managing surgical hardware, in accordance with a first variant;

FIG. 4 is a flow chart of a method for managing surgical hardware, in accordance with a second variant;

FIG. 5 is an image of a sterilization tray as used in the methods of the present disclosure; and

FIG. 6 is an image of a storage device as used in the method of the present disclosure.

### DETAILED DESCRIPTION

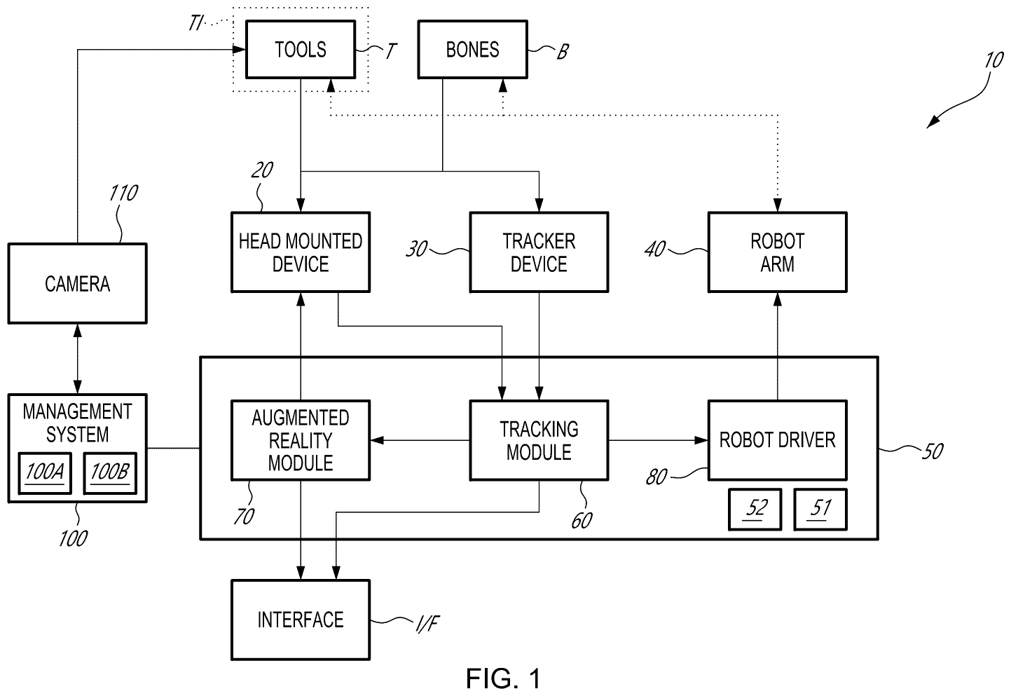

Referring to the drawings and more particularly to FIG. 1, computer-assisted surgery (CAS) system with optional head-mounted navigation with optional tracking is generally shown at 10, and is used to provide surgical assistance to an operator. The CAS system 10 may be used jointly with a system for managing surgical hardware 100, referred to as management system 100 as well. While the CAS system 10 and the system for management of surgical hardware 100 are shown as being discrete elements in FIG. 1, the system for management of surgical hardware 100 could be integrated into the CAS system 10. Likewise, the system for management of surgical hardware 100 could be used independently of the CAS system 10. The CAS system 10 may be used to assist an operator wearing a head-mounted device with display in performing the afore-mentioned maneuvers and/or other surgical maneuvers on a patient, such as surgical maneuvers associated with orthopedic surgery, including pre-operative analysis of range of motion, and implant assessment planning, as described hereinafter.

The CAS system 10 may be robotized in a variant, and has, may have or may be used with a head-mounted device or tracking device 20, a tracking device 30, a robot arm 40, a CAS controller 50, a tracking module 60, an augmented reality module 70, and a robot driver 80, or any combination thereof:
The head-mounted device 20 is worn by an operator, such as by the surgeon performing surgery, and may be referred to as head-mounted assistance device 20 as it has the capacity to capture images, such as in video format. The head-mounted device 20 may have a display screen to provide data to the wearer, though this may be optional in an embodiment. For simplicity, the expressions head-mounted assistance device 20 and head-mounted device 20 are used interchangeably in the present disclosure and figures. The head-mounted assistance device 20 may be used to provide a display in augmented/mixed and/or virtual reality to a user. The head-mounted assistance device 20 may also be tasked with taking images of the surgery, with the images being used for the tracking of patient tissue (such as bones) and tools, for instance as a video feed. The head-mounted assistance device 20 may also be used as an interface by which an operator may communicate commands to the CAS system 10, and/or to the system for management of surgical hardware 100.
As an alternative to the head-mounted device 20, the tracking device 30 may optionally be used to track the patient tissue, instruments, and the robot arm 40. For example, the tracking device 30 may complement the tracking performed with the imaging done with the head-mounted assistance device 20, and may hence be referred to as a secondary tracking device. The tracking device 30 may employ camera technology similar to that of the head-mounted assistance device 20, such as depth cameras, with optional pattern projector, as described below, or may be a different imaging technology, to provide its video feed. These various cameras may be referred to as non-radiographic cameras, digital cameras, etc. The tracking device 30 may be said to be stationary. The tracking device 30 may be the primary tracking device 30, if the head-mounted assistance device 20 is absent or temporarily out of the line of the surgical scene, or with the head-mounted assistance device 20 providing complementary tracking capability. The tracking device 30 may use ultrasounds as well. The tracking device 30 may operate with optical trackers on the objects, such as those used in a Navitrack® system, but other types of trackers could be used, including QR codes, AprilTags, 3D trackers having a wider range of visibility (cube or other prism), etc.
The robot arm 40 may optionally be present as the working end of the system 10—the CAS system 10 could also be non robotic—, and may be used to guide or to perform bone alterations as planned by an operator and/or the CAS controller 50 and as controlled by the CAS controller 50, or may be used to support a tool T that is operated by a surgeon. The robot arm 40 may also be configured for collaborative/cooperative mode in which the operator may manipulate the robot arm 40. For example, the tooling end, also known as end effector, and/or tool T at the tooling end may be manipulated by the operator while supported by the robot arm 40;
The CAS controller 50 includes the processor(s) and appropriate hardware and software to run a computer-assisted surgery procedure in accordance with one or more workflows. The CAS controller 50 may include or operate the tracking module 60, the augmented reality module 70, and/or the robot driver 80 if present. Moreover, as described hereinafter, the CAS controller 50 may also drive the robot arm 40 through a planned surgical procedure, if the robot arm 40 is present;
The tracking module 60 is tasked with determining the position and/or orientation of the various relevant objects during the surgery procedure, such as the bone(s) and tool(s), using data acquired by the head-mounted assistance device 20 (e.g., video feed) if present, the tracking device 30 if present. The position and/or orientation may be used by the CAS controller 50 to control the robot arm 40;
The augmented reality module 70 (a.k.a., mixed reality module) is provided to produce an augmented reality output to the operator, for instance for display in the head-mounted assistance device 20. The augmented reality module 70 may also produce other types of outputs, including a virtual reality output. The augmented reality module 70 may provide its output to displays other than head-mounted or wearable displays. For example, the augmented reality module 70 may produce an output for display on monitors of the CAS system 10, shown in FIG. 1 as interface I/F;
The robot driver 80 is tasked with powering or controlling the various joints of the robot arm 40, if present, based on operator demands or on surgery planning.

Other components, devices, systems, may be present, such as surgical instruments and tools T, the interfaces I/F such as displays, screens, computer station, servers, and the like etc.

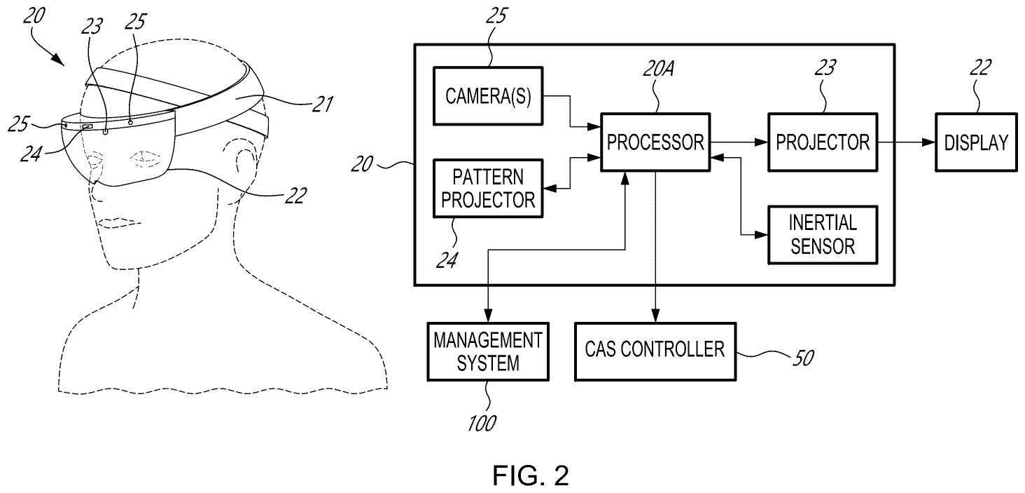

Referring to FIG. 2, a schematic example of the head-mounted assistance device 20 is provided. The head-mounted assistance device 20 may be as described in U.S. Pat. No. 10,687,568, the contents of which are incorporated herein by reference, or may have other configurations. The head-mounted assistance device 20 described as a surgical helmet assembly in U.S. Pat. No. 10,687,568 is well suited to be used in an augmented reality setting by its configuration. The head-mounted assistance device 20 may have a head enclosure 21 shaped to encircle a head of an operator. The head enclosure 21 may be straps, a rim, a helmet, etc. A face shield 22 may be mounted to a forehead or brow region of the head enclosure 21. The face shield 22 may be transparent to allow see-through vision by a user, but with the option of serving as a screen for augmented reality. Other components of the head-mounted assistance device 20 may include stabilizers, head band, a ventilation system with fan and vents, a light source, a rechargeable power source (e.g., a battery) etc.

The head-mounted assistance device 20 may consequently include a processor 20A and components to produce a mixed reality session. For instance, the head-mounted assistance device 20 may have an integrated projector 23 that may project data on the face shield 22, in a manner described below. Alternatively, the face shield 22 may be a screen having the ability to display images. As an example, the head-mounted assistance device 20 may be a HoloLens®. In an embodiment, the face shield 22 is a display-like unit of the type that may be used in virtual reality, with camera(s) therein to create a mixed reality output using camera footage, such as an Oculus Rift®, smartphone with head support, etc, hologram projection in augmented reality. The head-mounted assistance device 20 may include one or more orientation sensors, such as inertial sensor unit(s) (e.g., shown as 30), for an orientation of the head-mounted assistance device 20 to be known and tracked.

According to an embodiment, the head-mounted assistance device 20 is equipped to perform optical tracking of an implant IN, such as the intramedullary implant or orthopedic plate, patient tissue B, instruments T and/or robot arm 40, from a point of view (POV) of the operator. The head-mounted assistance device 20 may therefore have one or more imaging devices or apparatuses, to capture video images of a scene, i.e., moving visual images, a sequence of images over time. In a variant, the video images are light backscatter (a.k.a. backscattered radiation) used to track objects. In the present disclosure, the head-mounted assistance device 20 may be used to track tools and bones so as to provide navigation data in mixed reality to guide an operator based on surgery planning. Backscattered radiation can also be used for acquisition of 3D surface geometries of bones and tools.

The head-mounted assistance device 20 may produce structured light illumination for tracking objects with structured light 3D imaging. In structured light illumination, a portion of the objects is illuminated with one or multiple patterns from a pattern projector 24 or like light source. The pattern projector 24 includes infrared light projection. Structured light 3D imaging is based on the fact that a projection of a line of light from the pattern projector 24 onto a 3D shaped surface produces a line of illumination that appears distorted as viewed from perspectives other than that of the pattern projector 24. Accordingly, imaging such a distorted line of illumination allows a geometric reconstruction of the 3D shaped surface. Imaging of the distorted line of illumination is generally performed using one or more cameras 25 (including appropriate components such as e.g., lens(es), aperture, image sensor such as CCD, image processor) which are spaced apart from the pattern projector 24 so as to provide such different perspectives, e.g., triangulation perspective. In some embodiments, the pattern projector 24 is configured to project a structured light grid pattern including many lines at once as this allows the simultaneous acquisition of a multitude of samples on an increased area. In these embodiments, it may be convenient to use a pattern of parallel lines. However, other variants of structured light projection can be used in some other embodiments.

The structured light grid pattern can be projected onto the surface(s) to track using the pattern projector 24. In some embodiments, the structured light grid pattern can be produced by incoherent light projection, e.g., using a digital video projector, wherein the patterns are typically generated by propagating light through a digital light modulator. Examples of digital light projection technologies include transmissive liquid crystal, reflective liquid crystal on silicon (LCOS) and digital light processing (DLP) modulators. In these embodiments, the resolution of the structured light grid pattern can be limited by the size of the emitting pixels of the digital projector. Moreover, patterns generated by such digital display projectors may have small discontinuities due to the pixel boundaries in the projector. However, these discontinuities are generally sufficiently small that they are insignificant in the presence of a slight defocus. In some other embodiments, the structured light grid pattern can be produced by laser interference. For instance, in such embodiments, two or more laser beams can be interfered with one another to produce the structured light grid pattern wherein different pattern sizes can be obtained by changing the relative angle between the laser beams.

The pattern projector 24 may emit light that is inside or outside the visible region of the electromagnetic spectrum. For instance, in some embodiments, the emitted light can be in the ultraviolet region and/or the infrared region of the electromagnetic spectrum such as to be imperceptible to the eyes of the medical personnel. In these embodiments, however, the medical personnel may be required to wear protective glasses to protect their eyes from such invisible radiations, and the face shield 22 may have protective capacity as well. As alternatives to structured light, the head-mounted assistance device 20 may also operate with laser rangefinder technology or triangulation, as a few examples among others.

The head-mounted assistance device 20 may consequently include the cameras 25 to acquire backscatter images of the illuminated portion of objects. Hence, the cameras 25 capture the pattern projected onto the portions of the object. The cameras 25 are adapted to detect radiations in a region of the electromagnetic spectrum that corresponds to that of the patterns generated by the light projector 24. As described hereinafter, the known light pattern characteristics and known orientation of the pattern projector 24 relative to the cameras 25, are used by the tracking module 60 to generate a 3D geometry of the illuminated portions, using the backscatter images captured by the camera(s) 25. Although a single camera spaced form the pattern projector 24 can be used, using more than one camera 25 may increase the field of view and increase surface coverage, or precision via triangulation. The head-mounted assistance device 20 is shown as having a pair of cameras 25 is used.

The head-mounted assistance device 20 may also have one or more filters integrated into either or both of the cameras 25 to filter out predetermined regions or spectral bands of the electromagnetic spectrum. The filter can be removably or fixedly mounted in front of any given camera 25. For example, the filter can be slidably movable into and out of the optical path of the cameras 25, manually or in an automated fashion. In some other embodiments, multiple filters may be periodically positioned in front of a given camera in order to acquire spectrally resolved images with different spectral ranges at different moments in time, thereby providing time dependent spectral multiplexing. Such an embodiment may be achieved, for example, by positioning the multiple filters in a filter wheel that is controllably rotated to bring each filter in the filter wheel into the optical path of the given one of the camera 25 in a sequential manner.

In some embodiments, the filter can allow transmittance of only some predetermined spectral features of objects within the field of view, captured either simultaneously by the head-mounted assistance device 20 or separately by the secondary tracking device 90, so as to serve as additional features that can be extracted to improve accuracy and speed of registration.

More specifically, the filter can be used to provide a maximum contrast between different materials which can improve the imaging process and more specifically the soft tissue identification process. For example, in some embodiments, the filter can be used to filter out bands that are common to backscattered radiation from typical soft tissue items, the surgical structure of interest, and the surgical tool(s) such that backscattered radiation of high contrast between soft tissue items, surgical structure and surgical tools can be acquired. Additionally, or alternatively, where white light illumination is used, the filter can include band pass filters configured to let pass only some spectral bands of interest. For instance, the filter can be configured to let pass spectral bands associated with backscattering or reflection caused by the bones, the soft tissue while filtering out spectral bands associated with specifically colored items such as tools, gloves and the like within the surgical field of view. Other methods for achieving spectrally selective detection, including employing spectrally narrow emitters, spectrally filtering a broadband emitter, and/or spectrally filtering a broadband imaging detector (e.g., the camera 25), can also be used. Another light source may also be provided on the head-mounted assistance device 20, for a secondary tracking option, as detailed below. It is considered to apply distinctive coatings on the parts to be tracked, such as the bone and the tool, to increase their contrast relative to the surrounding soft tissue.

In accordance with another embodiment, the head-mounted assistance device 20 may include a 3D camera(s), also shown as 25, to perform range imaging, and hence determine position data from the captured images during tracking-FIG. 2 showing two of such cameras 25 to enhance a depth perception. The expression 3D camera is used to describe the camera's capability of providing range data for the objects in the image or like footage it captures, but the 3D camera may or may not produce 3D renderings of the objects it captures. In contrast to structured light 3D imaging, range tracking does not seek specific illumination patterns in distance calculations, but relies instead on the images themselves and the 3D camera's capacity to determine the distance of points of objects in the images. Stated differently, the 3D camera for ranging performs non-structured light ranging, and the expression “ranging” is used herein to designate such non-structured light ranging. Such range tracking requires that the 3D camera be calibrated to achieve suitable precision and accuracy of tracking. In order to be calibrated, the head-mounted assistance device 20 may use a known visual pattern in a calibration performed in situ, at the start of the tracking, and optionally updated punctually or continuously throughout the tracking. The calibration is necessary to update the camera acquisition parameters due to possible lens distortion (e.g., radial, rotational distortion), and hence to rectify image distortion to ensure the range accuracy. Moreover, as described herein, tracking tokens with recognizable patterns (e.g., QR codes) may be used, with the patterns being used to determine a point of view (POV) of the cameras 25 of the head-mounted assistance device 20, via perspective deformation.

In a variant, the head-mounted assistance device 20 only has imaging capacity, for instance through cameras 25 (of any type described above), optionally pattern projector 24, without other components, such as face shield 22, etc.

Referring to FIGS. 1 and 2, if present, the robot arm 40 may stand from a base, for instance in a fixed relation relative to the operating-room (OR) table supporting the patient, whether it is attached or detached from the table. The robot arm 40 has a plurality of joints and links, of any appropriate form, to support a tool T that interfaces with the patient. The end effector or tool head may indeed optionally incorporate a force/torque sensor for collaborative/cooperative control mode, in which an operator manipulates the tool T at the end of the robot arm 40. The robot arm 40 is shown being a serial mechanism, arranged for the tool head to be displaceable in a desired number of degrees of freedom (DOF). For example, the robot arm 40 controls 6-DOF movements of the tool head, i.e., X, Y, Z in the coordinate system, and pitch, roll and yaw. Fewer or additional DOFs may be present. For simplicity, only a generic illustration of the joints and links is provided, but more joints of different types may be present to move the tool head in the manner described above. The joints are powered for the robot arm 40 to move as controlled by the CAS controller 50 in the six DOFs, and in such a way that the position and orientation of the tool head in the coordinate system may be known, for instance by readings from encoders on the various joints. Therefore, the powering of the joints is such that the tool head of the robot arm 40 may execute precise movements, such as moving along a single direction in one translation DOF, or being restricted to moving along a plane, among possibilities. Such robot arms 40 are known, for instance as described in U.S. patent application Ser. No. 11/610,728, and incorporated herein by reference. The head-mounted assistance device 20 and/or tracking device 30 may be used for the tracking of the end effector of the robot arm 10, or other systems such as inertial sensor systems, e.g., an inertial sensor unit may be on the robot arm 40.

Still referring to FIG. 1, the CAS controller 50 is shown in greater detail relative to the other components of the robotized CAS system 10. The CAS controller 50 has a processor unit 51 (one or more processors) and a non-transitory computer-readable memory 52 communicatively coupled to the processing unit 51 and configured for executing computer-readable program instructions executable by the processing unit 51 to perform some functions, such as tracking the patient tissue and tools, using the camera feed from the head-mounted assistance device 20 and/or from the tracking device 90, and the readings from the inertial sensor unit(s) 30. The CAS controller 50 may also control the movement of the robot arm 40. The CAS system 10 may comprise various types of interfaces I/F, for the information to be provided to the operator. In addition to the head-mounted assistance device 20, the interfaces I/F may include a monitor and/or screens including wireless portable devices (e.g., phones, tablets), audio guidance, LED displays, among many other possibilities. For example, the interface D includes a graphic-user interface (GUI) operated by the system 10. The CAS controller 50 may also display images captured by the cameras 25 of the head-mounted assistance device 20 and/or tracking device 30, for instance to be used in the collaborative/cooperative control mode of the system 10, or for visual supervision by the operator of the system 10, with augmented reality for example. The CAS controller 50 may drive the robot arm 40, if present, in performing the surgical procedure based on the surgery planning achieved pre-operatively. The CAS controller 50 may run various modules, in the form of algorithms, code, non-transient executable instructions, etc, in order to operate the CAS system 10 in the manner described herein. The CAS controller 50 may be part of any suitable processor unit(s), such as a personal computer or computers including laptops and desktops, tablets, server, cloud, etc.

The tracking module 60 may be a subpart of the CAS controller 50, or an independent module or system. The tracking module 60 receives from the head-mounted assistance device 20 and the tracking device 90 (if present) the video feed of the surgical scene, e.g., as backscatter images of the objects. The tracking module 60 may also concurrently receive tracking data (e.g., orientation data) from the inertial sensor unit(s) 30. In an embodiment, as the system 10 performs real-time tracking, the video images and the orientation data are synchronized, as they are obtained and processed simultaneously. Other processing may be performed to ensure that the video footage and the orientation data are synchronized.

The tracking module 60 processes the video images to track one or more objects, such as a bone, an instrument, etc. The tracking module 60 may determine the relative position of the objects, and segment the objects within the video images. In a variant, the tracking module 60 may process the video images to track a given portion of an object, that may be referred to as a landmark. The landmark may be different parts of the objects, objects on the objects, such as tracking tokens with recognizable patterns, etc.

The tracking module 60 may also be provided with models of the objects to be tracked. For example, the tracking module 60 may track bones, implants IN and tools, and hence uses virtual bone models and tool models. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc). The bone models may therefore be patient specific. It is also considered to obtain bone models from a bone model library, with the data obtained from the video images used to match a generated 3D surface of the bone with a bone from the bone atlas. The virtual implant models and/or tool models may be provided by the implant/tool manufacturer, or may also be generated in any appropriate way so as to be a virtual 3D representation of the tool(s).

In a variant, the tracking module 60 may generate 3D models using the video images. For example, if the tracking module 60 can have video images of a tool, from 360 degrees, it may generate a 3D model that can be used for subsequent tracking. This intraoperative model may or may not be matched with pre-existing or pre-operative model of the tool and/or of the implant.

Additional data may also be available, such as tool orientation (e.g., axis data and geometry). By having access to bone, implant and tool models, the tracking module 60 may recognize an object in the image processing and/or may obtain additional information, such as the axes related to bones or tools. The image processing by the tracking module 60 may be assisted by the presence of the models, as the tracking module 60 may match objects from the video images with the virtual models.

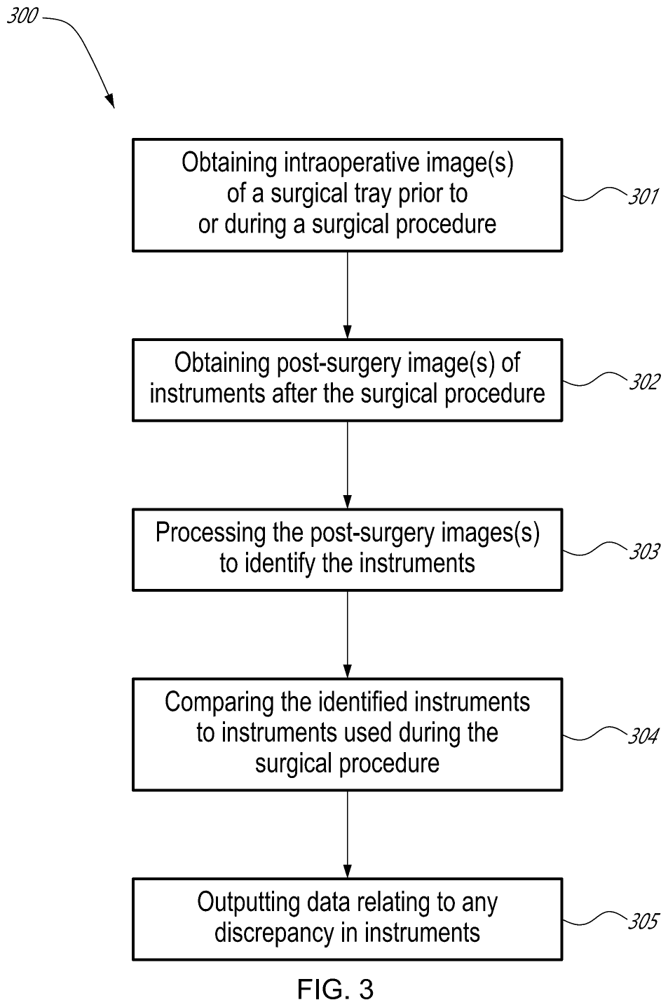

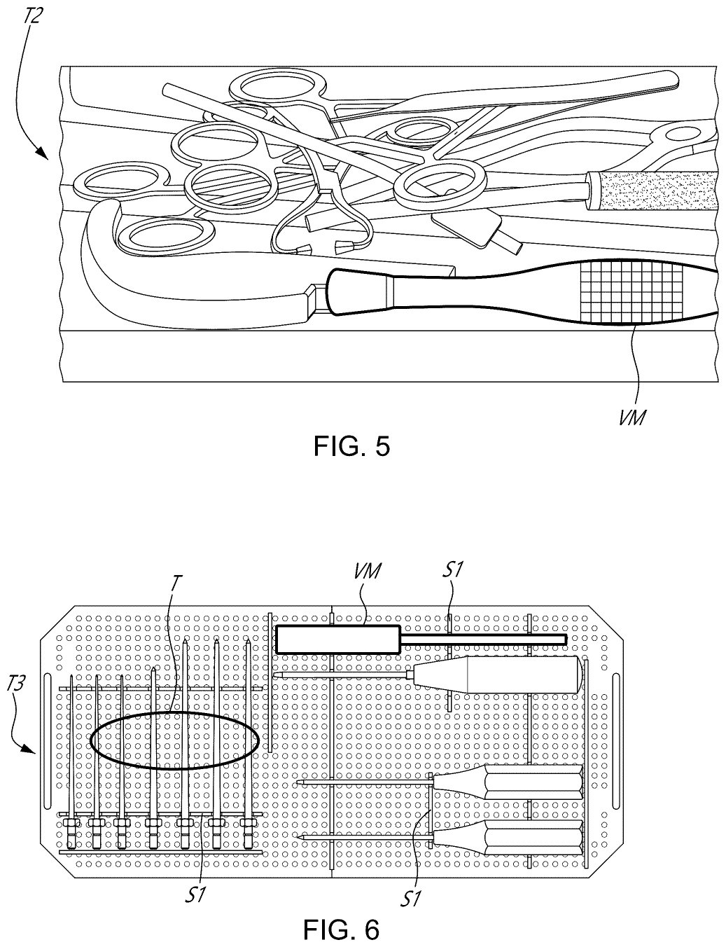

Referring to FIGS. 1 and 2, the system for management of surgical hardware 100 is shown as being used jointly with the CAS system 10 and the head-mounted assistance device 20. The system for management of surgical hardware 100 may be used alone, such as with camera 110. The camera 110 may be non-radiographic cameras of any type, a depth camera, a digital camera, etc. The system for management of surgical hardware 100 may include a processing unit 100A, and a non-transitory computer-readable memory 100B that may be communicatively coupled to the processing unit and comprising computer-readable program instructions executable by the processing unit 100A. The system for management of surgical hardware 100 may be used in order to assist in managing the surgical hardware, such as shown as the tools T in FIG. 1. This may include any physical tool that may be used during the surgical procedure. In a variant, the tools T are sterilized and placed on a surgical tray T1. The surgical tray T1 may enter the sterile zone during surgery, and may be in close proximity to the surgeon and staff, for proximate access. The tools T may come from storage devices T2, such as shown in FIG. 6. In a variant, there may be a plurality of the surgical tray T1 used in a single surgical procedure for a single patient. The surgical tray T1 may be similar to the one shown as T3 in FIG. 6, in which the various tools T each have a predetermined and set location in the tray T3, i.e., each tool T may have a given and planned position in the tray T3. For example, the tray T3 may have tool supports S1 specifically shaped and configured to hold respective tools T. In a variant, the tools T1 are captive in the tool supports S1. The tray T3 is shown as having a perforated plate (a.k.a., grid plate). The tray T3 may be configured to be sterilized, such as with the tools T1 therein. The optional perforated plate is well suited for sterilization, for instance to allow a sterilization fluid to pass through it to sterilize the tools T1. Thus, in a variant, the tools T are provided to the surgical team in the operating room in tray T1 (that may be embodied by tray T3 of FIG. 6), all of which are in a sterile state. The tools T may be reinstalled in the tray T3 after use yet intraoperatively, whether by robot manipulation or by hand. The tray T3 having used and unused tools T may then be subjected to sterilization in the state shown in FIG. 3, with used and unused referring to use during the surgical procedure. Alternatively, as described below with reference to FIG. 5, a tray of used tools T2 may be used, in which the tools T2 may be disposed of in random order.

The system for management of surgical hardware 100 may be operated in different ways, notably to assist in ensuring that tool inventory is preserved. With reference to FIG. 3, a method for managing surgical hardware is shown at 300, and may be operated by the system for management of surgical hardware 100, via its processing unit 100A and non-transitory computer-readable memory 100B, including through cloud computing.

In a variant, the starting point of the method 300 may be at the outset of surgery, referred to herein as intraoperative. This may include the peri-operative phase, in which the materials and facilities are being prepared for a surgical procedure, and the operative phase, during which the patient is undergoing surgery.

According to step 301, one or more intraoperative images of a surgical tray are obtained, prior to or during the surgical procedure. The surgical tray may be as shown at T1 in FIG. 1 and/or as T3 in FIG. 6. Step 301 is optional, and may also include obtaining images of the storage device, such as shown at T3 in FIG. 6. Obtaining images may include taking images, such as by camera 110, or by the cameras of the CAS system 10, such as that of the head-mounted assistance device 20 and/or the tracker device 30. The intraoperative image(s) may be processed to identify the instruments on the surgical tray. The processing of images herein may include identifying a contour of an instrument, scaling the instrument and estimating its dimensions, comparing the image of the instrument with a database of other instruments, to identify the object. In a variant, if the system for management of surgical hardware 100 has access to a brand of instruments, the database may be provided for the brand and/or type of procedure. The processing of images may also include identifying any code (e.g., text, barcode, QR code) or information on the object (e.g., logo). This may apply to all processing of images described herein. After the image processing, a list of instruments on the surgical tray T1 or T3 may be generated. In a variant of step 301, or as a supplemental step, the system may obtain a manufacturer file, proprietary file or like identification file of a tray T1 and T3, or equivalent, in which the tray T1 or T3 and the tools T are imaged (virtually or by photograph) or modelled in a 3D digital model, for instance with each tool T in its set position in the tray T1 or T3, the set position being the position where a tool is expected to be positioned (e.g., the home position) as the support S1 at the set position may be specifically shaped to receive the tool. If the system has access to such a file in supplement to the intraoperative images, the system may perform a verification step to ensure that the intraoperative images match the file, to confirm or infirm a completeness of the tray T1 or T3.

According to step 302, one or more post-surgery images of a plurality of instruments used in a surgical procedure is obtained, after the surgical procedure is completed and before the plurality of instruments are stored in a storage device, such as the storage device T2 of FIG. 5, or with the instruments in the storage tray T1 or T3 (FIG. 6) after use, for example if the operator or robot repositions the instruments in their respective positions in the tray T3. In a variant, step 302 is the first step performed by the system for management of surgical hardware 100, as step 301 and its substeps are optional. Obtaining one or more post-surgery images may include obtaining a video feed of the plurality of instruments, such as to have numerous points of view of the instruments. The video feed may be separated in stills, to contribute to the processing. In a variant, the post-surgery images are obtained when the plurality of instruments are in a sterilization tray, bucket, etc, prior to sterilization. For example, one such tray with instruments is shown in as T3 in FIG. 6. However, the post-surgery images may be obtained after sterilization as well, or alternatively. Step 302 may include imaging the instruments. For example, this may be done by camera 110 associated with the system for management of surgical hardware 100. The camera 110 may be a non-radiographic digital camera. In a variant, the camera 110 is a depth camera or equivalent that can generate a model of the instrument or a part thereof. The model may be a three-dimensional virtual representation, for example, to scale (i.e., with dimensioning).

According to step 303, the post-surgery image(s) is(are) processed to identify the instruments of the plurality. More particularly, an identity of each instrument may be determined. The processing of images herein may include identifying a contour of an instrument, scaling the instrument and estimating its dimensions, comparing the image of the instrument with a database of other instruments, to identify the object. In a variant, if the system for management of surgical hardware 100 has access to a brand of instruments, the database may be provided for the brand and/or type of procedure. The processing of images may also include identifying any code (e.g., text, barcode, QR code) or information on the object (e.g., logo). This may thus include recognizing an optical code on one or more of the instruments when processing the post-surgery images(s) to identify the instruments of the plurality, and retrieving the identity of the instrument from the optical code. The database of other instruments may include three-dimensional models of instruments (i.e., 3D virtual representations). Accordingly, the post-surgery image(s) is(are) processed to identify the instruments of the plurality, by comparing three-dimensional models, e.g., by overlaying. Hence, the image processing may include geometric comparison of imaged model(s) with database model(s).

According to step 304, the instruments from the image processing of step 303 is compared with a list of instruments used during the surgical procedure. In a variant, the list of instruments may be obtained from a computer-assisted surgery system operating a surgical workflow during the surgical procedure, such as the CAS system 10. The list of instruments may have been obtained via step 301 as another option. As an option, in step 304, the list of instruments may be compared to the set position in the tray, for example with reference to the tray T3 of FIG. 6, which tray T3 may be used as tray T1 and also as sterilization tray.

According to step 305, data relating to any discrepancy between the list and the plurality of instruments may be output. For example, the data may identify any missing instrument, any damaged instrument, any instrument that should not be present but is present, any instrument misplaced in the tray (e.g., tray T3 in FIG. 6). The data may take various form, such as text describing instruments, with identification number, and/or a description of the instrument, images of the instrument. The method 300 may therefore be used to ensure that the instrument/tool set is complete and/or that the instruments are correctly positioned in their set positions in the tray. Because of the multiple staff involved in the various steps of manipulating, sterilizing, storing, the method 300 is automated and can ensure that missing instruments are identified, in spite of the multiple staff involved.

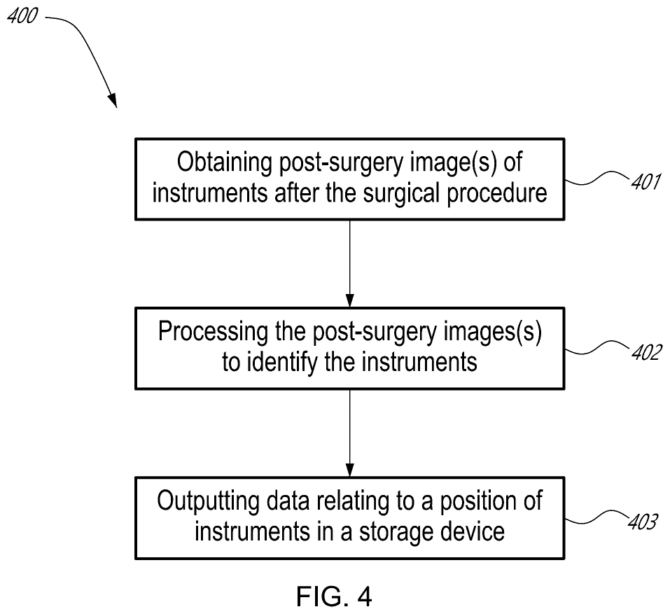

Referring now to FIG. 4, the system for management of surgical hardware 100 may be operated in another way, to assist in managing the surgical hardware in storage. In FIG. 4, a method for managing surgical hardware is shown at 400, and may be operated by the system for management of surgical hardware 100, via its processing unit 100A and non-transitory computer-readable memory 100B, including through cloud computing.

According to step 401, one or more post-surgery image(s) of a plurality of instruments used in a surgical procedure is(are) obtained, after the surgical procedure is completed and before the plurality of instruments are stored in a storage device, such as the storage device T3 of FIG. 6. Obtaining one or more post-surgery images may include obtaining a video feed of the plurality of instruments, such as to have numerous points of view of the instruments. The video feed may be separated in stills, to contribute to the processing. In a variant, the post-surgery images are obtained when the plurality of instruments are in a sterilization tray, bucket, etc, prior to sterilization. For example, one such tray with instruments is shown in as T2 in FIG. 5, in the form of a disposal tray, and as T3 in FIG. 6, in which the instruments have a set position. However, the post-surgery images may be obtained after sterilization as well, or alternatively. Step 401 may include imaging the instruments. For example, this may be done by camera 110 associated with the system for management of surgical hardware 100. The camera 110 may be a non-radiographic digital camera. The camera 110 may be part of a head-mounted device, such as 20 in FIG. 1, whereby imaging the plurality of instruments with the non-radiographic digital camera may occur from a camera head-mounted to a head of a user. Again, in a variant, the camera 110 is a depth camera or equivalent that can generate a model of the instrument or a part thereof. The model may be a three-dimensional virtual representation, for example, to scale (i.e., with dimensioning).

According to step 402, the post-surgery image(s) is(are) processed to identify the instruments of the plurality. More particularly, an identity of each instrument may be determined. The processing of images herein may include identifying a contour of an instrument, scaling the instrument and estimating its dimensions, comparing the image of the instrument with a database of other instruments, to identify the object. In a variant, if the system for management of surgical hardware 100 has access to a brand of instruments, the database may be provided for the brand and/or type of procedure. The processing of images may also include identifying any code (e.g., text, barcode, QR code) or information on the object (e.g., logo). This may thus include recognizing an optical code on one or more of the instruments when processing the post-surgery images(s) to identify the instruments of the plurality, and retrieving the identity of the instrument using the optical code. The database of other instruments may include three-dimensional models of instruments (i.e., 3D virtual representations). Accordingly, the post-surgery image(s) is(are) processed to identify the instruments of the plurality, by comparing three-dimensional models, e.g., by overlaying. Hence, the image processing may include geometric comparison of imaged model(s) with database model(s).

According to step 403, data relating to a position of one or more of the instruments in the storage device is output to assist a user in storing the one or more instruments in the storage device, such as T3 in FIG. 6. Step 403 may include outputting an image of the at least one instrument when outputting the data. For example, an image of the instrument may be output as currently laid, with a visual marker on the image. This may also include outputting an image of the storage device with a visual marker on the position when outputting said data. FIGS. 5 and 6 show for example, a visual marker VM that may be displayed on images of the storage device T2, or while in the sterilization tray T3 (after sterilization), to guide a user in picking up the instrument, and storing it at the predetermined position in the storage device T2. In a variant, if the head-mounted device 20 is used, this may be achieved as part of a mixed reality image, the visual marker VM being added to the point of the wearer. In a variant, the data relating to a position of one or more of the instruments in the storage device is in the form of robot driving instructions provided to a robot, such as shown in FIG. 1. More particularly, the robot driver 80 may receive instructions to pick and place instruments from a given location to the set position of the instruments in the storage device, such as T3 in FIG. 6. Accordingly, the robot 40 may perform these pick and place maneuvers.

While method 400 pertains to the storage of the instruments in a predetermined in a storage device such as T3, it may also have inventory verification features. The method 400 may therefore include obtaining a list of the plurality of instruments from a computer-assisted surgery system operating a surgical workflow during the surgical procedure; comparing the identified instruments from the plurality to the list of instruments used during the surgical procedure; and outputting data relating to any discrepancy between the list and the plurality of instruments, and this may include indicating that one or more instruments are misplaced relative to a set position.

The method 400 contributes to the management of surgical hardware 400, limiting material loss, and allowing an orderly storage of the instruments.

In a variant, the system for management of surgical hardware 100 may be described as being a system for managing surgical hardware in computer-assisted surgery, that may have: a processing unit; and a non-transitory computer-readable memory communicatively coupled to the processing unit and comprising computer-readable program instructions executable by the processing unit for: obtaining at least one post-surgery image of a plurality of instruments used in a surgical procedure, after the surgical procedure is completed and before the plurality of instruments are stored in a storage device; processing the at least one post-surgery image to identify the instruments of the plurality; comparing the identified instruments from the plurality to a list of instruments used during the surgical procedure; and outputting data relating to any discrepancy between the list and the plurality of instruments.

In another variant, the system for management of surgical hardware 100 may be described as being a system for managing surgical hardware in computer-assisted surgery, that may have: a processing unit; and a non-transitory computer-readable memory communicatively coupled to the processing unit and comprising computer-readable program instructions executable by the processing unit for: obtaining at least one post-surgery image of a plurality of instruments used in a surgical procedure, after the surgical procedure is completed and before the plurality of instruments are stored in a storage device; processing the at least one post-surgery image to identify the instruments of the plurality; and outputting data relating to a position of at least one of the instruments in the storage device to assist a user in storing the at least one instrument.

The systems described herein may be used in conventional surgery, i.e., in which no computer is involved. However, the expression “computer-assisted surgery” is used herein for the systems for managing surgical hardware, as a processor or computer is necessary to perform the steps.

1. A system for managing surgical hardware in computer-assisted surgery, comprising:
a processing unit; and
a non-transitory computer-readable memory communicatively coupled to the processing unit and comprising computer-readable program instructions executable by the processing unit for:
obtaining at least one post-surgery image of a plurality of instruments used in a surgical procedure;
processing the at least one post-surgery image to identify the instruments of the plurality; and
outputting data relating to a position of at least one of the instruments in a storage device to assist a user in storing the at least one instrument.

2. The system according to claim 1, wherein the computer-readable program instructions are executable by the processing unit for obtaining a list of the plurality of instruments from a computer-assisted surgery system operating a surgical workflow during the surgical procedure.

3. The system according to claim 2, wherein the computer-readable program instructions are executable by the processing unit for comparing the identified instruments from the plurality to the list of instruments used during the surgical procedure.

4. The system according to claim 3, wherein the computer-readable program instructions are executable by the processing unit for outputting data relating to any discrepancy between the list and the plurality of instruments.

5. The system according to claim 3, wherein the computer-readable program instructions are executable by the processing unit for obtaining the at least one post-surgery image of the plurality of instruments used in the surgical procedure in the storage tray, and for outputting data relating to any discrepancy between a current position and a set position of at least one of the plurality of instruments.

6. The system according to claim 1, wherein the computer-readable program instructions are executable by the processing unit for outputting said data relating to the position of at least one of the instruments in the storage device as instructions for driving a robot configured to store the at least one instrument in the storage device.

7. The system according to claim 6, wherein the computer-readable program instructions are executable by the processing unit for driving the robot to store the at least one instrument in the storage device.

8. The system according to claim 1, wherein the computer-readable program instructions are executable by the processing unit for obtaining a video feed when obtaining the at least one post-surgery image of the plurality of instruments.

9. The system according to claim 8, wherein the computer-readable program instructions are executable by the processing unit for processing the video feed when processing the at least one post-surgery image.

10. The system according to claim 1, wherein the computer-readable program instructions are executable by the processing unit for obtaining the at least one post-surgery image of the plurality of instruments in a sterilization tray.

11. The system according to claim 10, wherein the computer-readable program instructions are executable by the processing unit for obtaining the at least one post-surgery image of the plurality of instruments in the sterilization tray prior to sterilization.

12. The system according to claim 10, wherein the computer-readable program instructions are executable by the processing unit for obtaining the at least one post-surgery image of the plurality of instruments in the sterilization tray after sterilization.

13. The system according to claim 10, wherein the computer-readable program instructions are executable by the processing unit for obtaining the at least one post-surgery image of the plurality of instruments in the sterilization tray, the sterilization tray being the storage device.

14. The system according to claim 1, wherein the computer-readable program instructions are executable by the processing unit for imaging the plurality of instruments to obtain the at least one post-surgery image.

15. The system according to claim 1, wherein the computer-readable program instructions are executable by the processing unit for imaging the plurality of instruments with a non-radiographic digital camera.

16. The system according to claim 1, wherein the computer-readable program instructions are executable by the processing unit for recognizing an optical code on at least one of the instruments when processing the at least one post-surgery image to identify the instruments of the plurality, and for retrieving the identity of the instrument from the optical code.

17. The system according to claim 1, wherein the computer-readable program instructions are executable by the processing unit for comparing images of the instruments to images of instruments in a database of instrument images when processing the at least one post-surgery image to identify the instruments of the plurality.

18. The system according to claim 1, wherein the computer-readable program instructions are executable by the processing unit for obtaining at least one post-surgery image of a plurality of instruments used in the surgical procedure after the surgical procedure is completed and before the plurality of instruments are stored in a storage device.

19. The system according to claim 1, wherein the computer-readable program instructions are executable by the processing unit for outputting an image of the at least one instrument when outputting said data.

20. The system according to claim 1, wherein the computer-readable program instructions are executable by the processing unit for outputting an image of the storage device with a visual marker on said position when outputting said data.
